(19) United States
(12) Patent Application Publication
(10) Pub. No.: US 2018/0116779 A1
(43) Pub. Date: May 3, 2018

(54) STERILE IMPLANT DELIVERY SYSTEM

(71) Applicant: Ann P. MARX, Wayne, IL (US)

(72) Inventor: Ann P. MARX, Wayne, IL (US)

(21) Appl. No.: 15/612,200

(22) Filed: Jun. 2, 2017

Related U.S. Application Data
(60) Provisional application No. 62/414,064, filed on Oct. 28, 2016.

(51) Int. Cl. A61F 2/00; A61F 2/12; A61B 50/30; A61F 2/02

(52) U.S. Cl. CPC A61F 2/0095; A61F 2/12; A61B 50/30; A61F 2240/00; A61B 2050/314; A61B 2050/3005; A61B 2050/3015; A61F 2/02

(57) ABSTRACT

Provided is a delivery system for inserting an implant into a surgical pocket, including a flexible sleeve having a first end and a second end and being sealed around its entire outer periphery forming an interior space, wherein the flexible sleeve is tapered toward at least one of the first end and the second end; and an implant contained and sealed within the interior space; in which the interior space and the implant contained and sealed therein are in a sterile condition. Further provided are a method of making the delivery system and a method of inserting an implant into a surgical pocket in a patient.

### CROSS-REFERENCE TO RELATED APPLICATION

The present application is related to and claims benefit under 35 U.S.C. § 119(e) to U.S. Provisional Application No. 62/414,064, filed 28 Oct. 2016, the entirety of which is incorporated herein by reference.

### TECHNICAL FIELD

The present invention relates to a delivery system for surgically implanting an implant prosthesis, particularly to a sterile implant delivery system.

### BACKGROUND

Various devices are known for delivery of an implant into a surgical pocket, including implantation of a flexible implant prosthesis, including, for example, a breast implant, a calf implant, a pectoral implant, a buttocks implant, a chin implant, a tissue expander, or other known flexible implant prosthetic devices. These devices are delivered separately and are assembled at the time of use in an operating theater. In general, the surgical theater is normally as sterile as possible, but it is nevertheless not unusual for sterility to be compromised during a surgical procedure involving many surgical personnel. Despite that many steps may have been taken to avoid loss of sterility and to provide antibiotics and other medicines to mitigate any such loss of sterility, problems resulting from non-sterile conditions continue in the surgical arts, particularly where long-term prosthetic devices are to be implanted.

### SUMMARY

The present invention addresses the problems of lack or loss of sterility of implanted flexible prosthetic devices, and provides for a significantly improved sterility of such flexible prosthetic devices.

Accordingly, in one embodiment, there is provided a delivery system for inserting an implant into a surgical pocket, including a flexible sleeve having a first end and a second end and being sealed around its entire outer periphery forming an interior space, wherein the flexible sleeve is tapered toward at least one of the first end and the second end; and an implant contained and sealed within the interior space; in which the interior space and the implant contained and sealed therein are in a sterile condition. In one embodiment, the implant contained and sealed within the flexible sleeve is provided from the manufacturer in a sterile condition, both externally and, particularly, internally, so that when it is opened for use, the entire contents of the device, including the implant, are sterile and can remain so throughout the surgical procedure. By providing the system in a sterile condition, the likelihood of contamination during the surgical implantation procedure is significantly reduced.

### BRIEF DESCRIPTION OF THE DRAWINGS

The present invention may be useful with a variety of types of surgically implanted devices. The annexed drawings are intended to provide a nonlimiting depiction of examples of an apparatus and to demonstrate the disclosed process, for the purpose of providing a better understanding of the invention, and are not intended to be limiting in any way. In the annexed drawings, like parts and features may have like or corresponding reference numerals. It should be appreciated that for simplicity and clarity of illustration, elements shown in the Figures have not necessarily been drawn to scale. For example, the dimensions of some of the elements may be exaggerated relative to each other for clarity.

FIG. 1 is a plan view of a delivery system for inserting an implant into a surgical pocket in accordance with an embodiment of the present invention.

FIG. 2 is a plan view of a delivery system for inserting an implant into a surgical pocket in accordance with another embodiment of the present invention.

FIG. 3 is a cross-sectional view of the embodiment of a delivery system for inserting an implant into a surgical pocket, taken at line A-A of FIG. 1.

FIG. 4 is a cross-sectional view of the embodiment of a delivery system for inserting an implant into a surgical pocket, taken at line B-B of FIG. 1.

FIGS. 5a, 5b and 5c are schematic depictions of steps in a method of making a delivery system for inserting an implant into a surgical pocket in accordance with an embodiment of the present invention.

FIG. 6 is a schematic depiction of a method of inserting an implant into a surgical pocket in a patient, in accordance with an embodiment of the present invention.

The foregoing Figures are provided as non-limiting examples of embodiments of the present invention, and are intended to facilitate understanding of the present invention.

### DETAILED DESCRIPTION

Surgical implants, and particularly breast implants, have been in use for over 40 years, and the implants and devices and methods for surgically inserting such implants have gradually evolved. However, some issues, as particularly relevant here, relating to possible contamination and the needed sterility of the implants, have remained. Recently, there has been a preference for minimal handling of implants during implantation, which is sometimes referred to as a “touchless technique”. The present invention includes the ability to provide for producing and shipping the implant packaged in a plastic bag or flexible sleeve that is suitable for use as a funnel for inserting the implant into a surgical pocket. In one embodiment, the flexible sleeve is manufactured with an implant-compatible lubricant within the bag. Thus, the implant is provided from the manufacturer already in a sealed bag forming a flexible sleeve with both the implant and at least the interior of the bag or flexible sleeve in a sterile condition. Of course, the entirety of the delivery system should be maintained in a sterile condition, both externally and internally. To facilitate this sterility, the delivery system is provided by the manufacturer in a suitably configured packaging designed to maintain the delivery system in a sterile condition. For example, implants may be placed in double packaging that ensures a dual microbiological barrier, to maintain sterility until implantation. The double packaging is delivered to the end user in a cardboard box that provides additional protection to provide the delivery system in the best possible condition.

In various embodiments, the flexible sleeve is shaped like a hexagon, a diamond, a cone, or a trapezoid shape, preferably in an elongated form. Other shapes may also be used, but it is considered that the hexagonal, diamond, trapezoidal or conical shapes, particularly any of these in an elongated form, are the most likely to be preferable. The shape should include at least one sufficiently tapered end to provide for cutting to form an opening of a suitable size for use as an outlet of the flexible sleeve, to create a funnel, for delivering the implant into a surgically formed pocket through the smallest possible incision. These features are described in more detail in the following description of various example embodiments of the invention.

In one embodiment, the flexible sleeve includes a valve or access port at one end of the bag, for example, on one side or end. The access port allows the surgeon or assistant to instill one or more of an antibiotic solution, an antibacterial, an antimicrobial, a lubricant, a gas, water, and/or a saline or other solution of the surgeon's choice into the bag. The valve or access port can be a needleless valve, or it can be a self-sealing seal suitable for penetration by a needle.

In one embodiment, on one or more corner or end of the bag are formed markings which show locations suitable for cutting to form an opening to modify the bag into a flexible sleeve that allows the implant to be squeezed out of the bag. In a process of implanting the device, the surgeon or assistant will cut the corner of the bag at the appropriate mark to allow the implant to be squeezed out of the bag without damaging the implant, place the opening of the bag into an incision forming a surgical pocket and then squeeze the flexible sleeve to thereby push the implant out of the flexible sleeve and into the surgical pocket. The flexible sleeve formed by cutting a corner or end off the sealed flexible sleeve will then be similar to the funnels and assist sleeves available in the prior art. Once the implant is in place in the surgical pocket, the bag is removed.

Thus, in one embodiment, the surgeon or assistant never directly touches the implant and the implant will never have touched the skin of the patient or any portion of the operating theater. The surgeon will of course need to use the usual care if the implant required adjustment within the surgical pocket so as not to contaminate the implant with skin or other flora that may be present.

Additional details relating to various embodiments of the present invention are set forth in the following detailed description.

In one embodiment, the delivery system for inserting an implant into a surgical pocket includes:

a flexible sleeve having a first end and a second end and being sealed around its entire outer periphery forming an interior space, wherein the flexible sleeve is tapered toward at least one of the first end and the second end; and

an implant contained and sealed within the interior space;

wherein the interior space and the implant contained and sealed therein are in a sterile condition. Of course, the outer surfaces of the delivery system should also be sterile, and the delivery system will normally be provided packaged within a sterile container, bag, envelope or other suitable packaging.

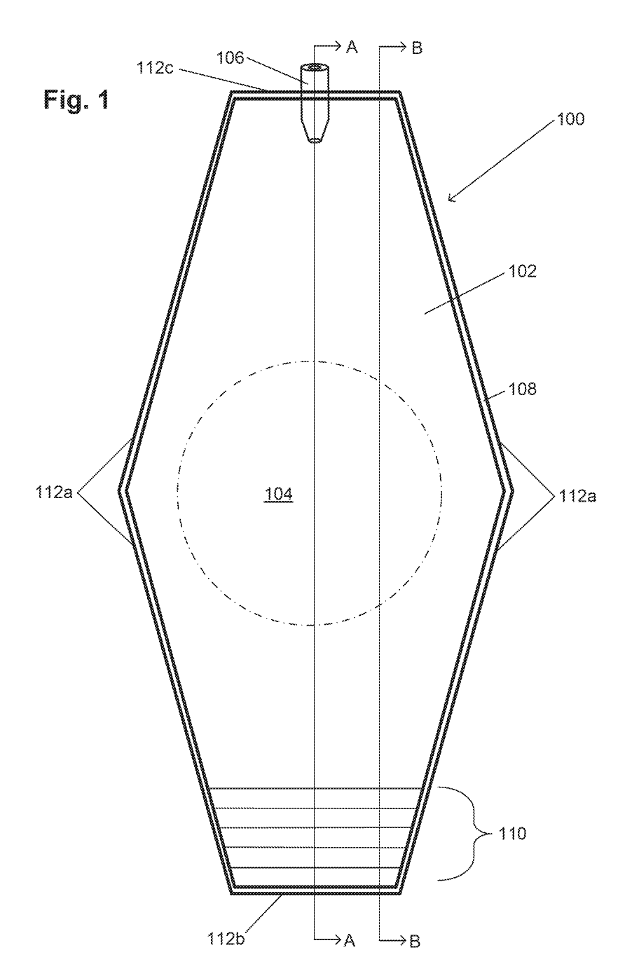

Referring now to FIG. 1, a delivery system 100 in accordance with an embodiment of the invention is shown. In one embodiment, the delivery system 100 includes a flexible sleeve 102 and, contained and sealed within the flexible sleeve 102, an implant 104. It is noted that the implant 104 is shown in dashed lines since it is enclosed and sealed within the interior space of the flexible sleeve 102.

The flexible sleeve 102 shown in FIG. 1 further includes a valve 106. The valve 106 is provided to enable the surgical team to insert or inject any desired material, such as one or more of an antibiotic solution, an antibacterial, an antimicrobial, a lubricant, a gas, water, or a saline or other solution, into the interior space of the flexible sleeve 102 while maintaining the sterility of the interior space and the implant 104. In one embodiment, the valve 106 may be a needleless valve, such as is used with IV equipment. In one embodiment, the valve 106 is not a needleless valve, but instead has a self-sealing closure, such as that on a vial for injection, in which the rubber seal can be penetrated by a needle, but self-seals when the needle is withdrawn. In one embodiment, the valve is optional, and may be omitted if all the materials that are desired to be used with the system 100 are already included within the flexible sleeve 102 at the time of manufacture. The valve 106 may be at any position around the periphery of the flexible sleeve, although it is shown in a specific position in FIG. 1. In one embodiment, the valve 106 may be placed in the end of the flexible sleeve that is to be cut off to form the opening through which the implant will be squeezed out of the flexible sleeve into the surgical envelope. This embodiment has the added benefit of removing the valve 106 from the flexible sleeve, which will avoid any possible leakage outward through the valve, resulting from the pressure applied to the flexible sleeve in forcing out the implant. In addition, removing the valve end in this embodiment would avoid any possible interference with the procedure by the valve.

As schematically depicted in FIG. 1 by a pair of or double lines 108 around the outer periphery of the flexible sleeve, the flexible sleeve 102 is sealed completely around its periphery during manufacture, so that the interior space of the flexible sleeve 102, including the implant 104 are contained and sealed within the flexible sleeve 102, and can be maintained in a sterile condition from the time of manufacture until the flexible sleeve is cut at one end to enable insertion of the implant 104 into a surgical pocket formed by the surgeon during a surgical procedure.

As shown in FIG. 1, in accordance with various embodiments of the present invention, the flexible sleeve 102 is marked with a plurality of lines 110, to assist the surgeon in selecting the appropriate place to cut the flexible sleeve 102 at the appropriate time at which the implant 104 is to be forced or squeezed out of the flexible sleeve 102 and into the surgical pocket formed in the patient's body for receiving the implant 104.

In the embodiment of FIG. 1, the flexible sleeve has an elongated hexagonal shape, in which four of the six sides, indicated by reference numeral 112a, are relatively longer, and two of the six sides, indicated by reference numerals 112b and 112 c, are relatively shorter, thus forming two end portions, one of which is useable as an exit port through which the implant 104 will be forced or squeezed out of the flexible sleeve 102 during the surgical procedure. As shown in FIG. 1, the short side 112b is adjacent the plurality of lines 110, while the short side 112c includes the valve 106. While it is intended in this embodiment that the short side 112b will be cut during the surgical procedure for removing the implant 104 from the flexible sleeve 102, it is also possible to cut the other short end 112c for removal of the implant 104, or for some other purpose desired by the surgeon.

It is noted that the embodiment depicted in FIG. 1 has six sides, all of which are straight, the longer sides 112a could be curved, either a smooth curve from end to end, or curved along some or all of the sides 112a. Similarly, the short ends 112b and 112c are shown as straight edges, but these edges could also be curved, within the scope of the present invention.

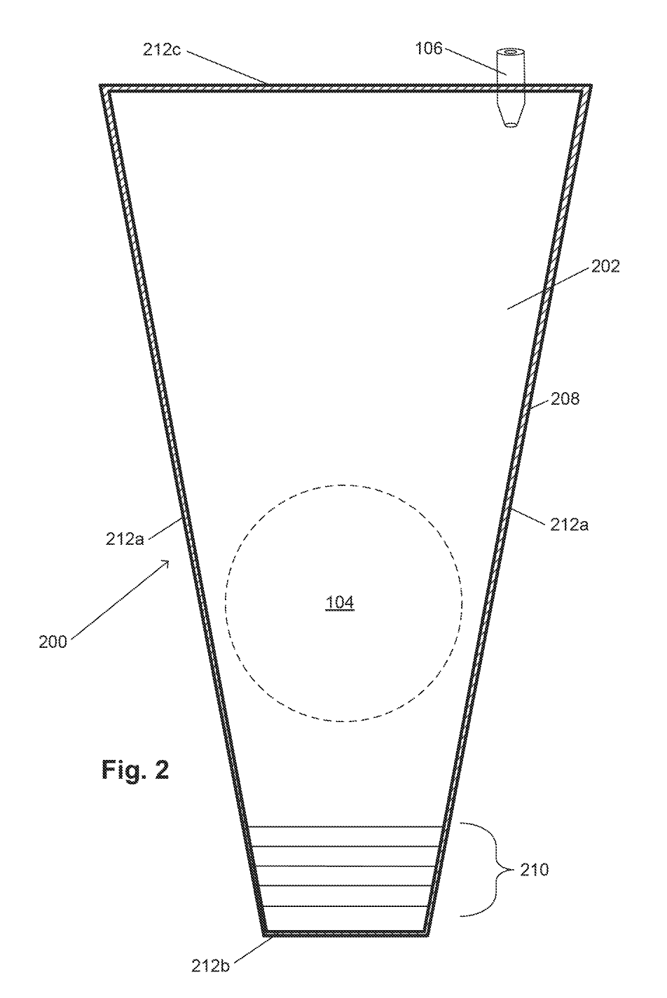

Referring now to FIG. 2, a delivery system 200 in accordance with an embodiment of the invention is shown. In one embodiment, the delivery system 200 includes a flexible sleeve 202 and, contained and sealed within the flexible sleeve 202, an implant 104. It is noted that the implant 104 is shown in dashed lines since it is enclosed and sealed within the interior space of the flexible sleeve 202.

The flexible sleeve 202 shown in FIG. 2 further includes a valve 106. The valve 106 is provided to enable the surgical team to insert or inject any desired material, such as one or more of an antibiotic solution, an antibacterial, an antimicrobial, a lubricant, a gas, water, or a saline or other solution, into the interior space of the flexible sleeve 202 while maintaining the sterility of the interior space and the implant 104. In one embodiment, the valve 106 may be a needleless valve, such as is used with IV equipment. In one embodiment, the valve 106 is not a needleless valve, but instead has a self-sealing closure, such as that on a vial for injection or other known self-sealing seal for a container, in which the rubber seal can be penetrated by a needle, but self-seals when the needle is withdrawn. In one embodiment, the valve is optional, and may be omitted if all the materials that are desired to be used with the system 200 are already included within the flexible sleeve 202 at the time of manufacture. The valve 106 may be at any position around the periphery of the flexible sleeve 202, although it is shown in a specific position in FIG. 2. In one embodiment, the valve 106 may be placed in the end of the flexible sleeve that is to be cut off to form the opening through which the implant will be squeezed out of the flexible sleeve into the surgical envelope. This embodiment has the added benefit of removing the valve 106 from the flexible sleeve, which will avoid any possible leakage outward through the valve, resulting from the pressure applied to the flexible sleeve in forcing out the implant. As schematically depicted in FIG. 2 by a pair of or double lines 208 around the outer periphery of the flexible sleeve, the flexible sleeve 202 is sealed completely around its periphery during manufacture, so that the interior space of the flexible sleeve 202, including the implant 104 are contained and sealed within the flexible sleeve 202, and can be maintained in a sterile condition from the time of manufacture until the flexible sleeve is cut at one end to enable insertion of the implant 104 into a surgical pocket formed by the surgeon during a surgical procedure.

As shown in FIG. 2, in accordance with various embodiments of the present invention, the flexible sleeve 202 is marked with a plurality of lines 210, to assist the surgeon in selecting the appropriate place to cut the flexible sleeve 202 at the appropriate time at which the implant 104 is to be forced or squeezed out of the flexible sleeve 202 and into the surgical pocket formed in the patient's body for receiving the implant 104.

In the embodiment of FIG. 2, the flexible sleeve has an elongated quadrangular shape, in which two of the four sides, indicated by reference numeral 212a, are relatively longer, and one of the four sides, indicated by reference numeral 212b is relatively short, and the fourth of the four sides, indicated by reference numeral 212 c, is of intermediate length, thus forming one end portion which is useable as an exit port through which the implant 104 will be forced or squeezed out of the flexible sleeve 202 during the surgical procedure. As shown in FIG. 2, the short side 212b is adjacent the plurality of lines 210, while the intermediate side 212c includes the valve 106. It is intended in this embodiment that the short side 212b to be cut during the surgical procedure for removing the implant 104 from the flexible sleeve 202. In this embodiment, it is also possible to cut the intermediate end 212c for some other purpose possibly desired by the surgeon.

It is noted that the embodiment depicted in FIG. 2 has four sides, all of which are straight, the longer sides 212a could be curved, either a smooth curve from end to end, or curved along some or all of the sides 212a. Similarly, the short end 212b and the intermediate end 212c are shown as straight edges, but these edges could also be curved, within the scope of the present invention.

In one embodiment, the flexible sleeve 102, 202 is formed of a material which is sufficiently flexible, but which is also sufficiently strong to withstand the pressures applied during use, and which also enables portions of the flexible sleeve 102, 202 to be flattened as much as possible, considering that the implant 104 is contained and sealed within the flexible sleeve 102, 202 during shipping and storage prior to use.

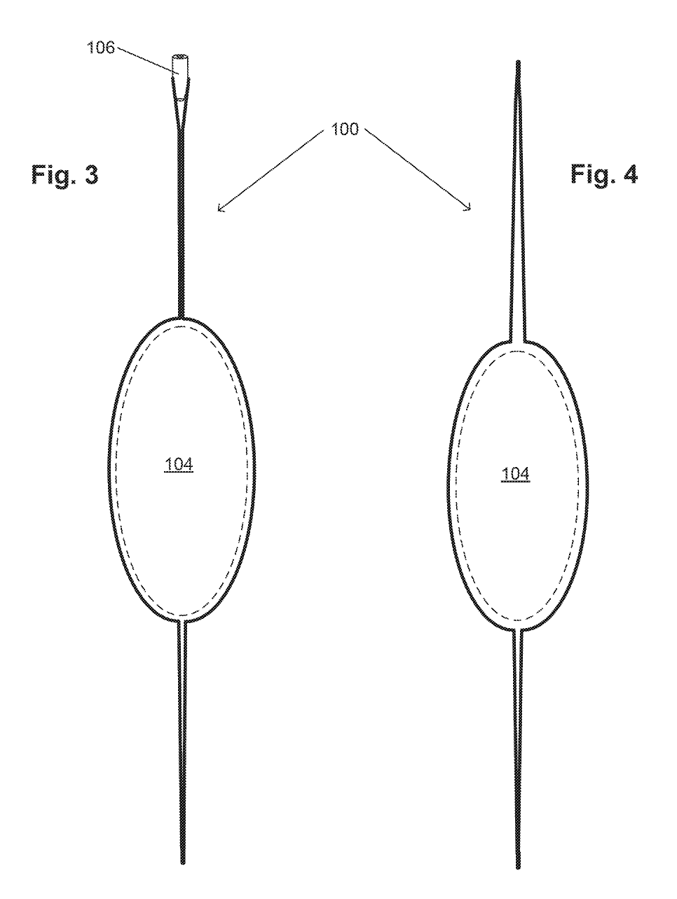

Referring now to FIGS. 3 and 4, these are cross-sectional views taken and lines A-A and B-B, respectively, as shown in FIG. 1. FIGS. 3 and 4 very schematically depict the flexible sleeve 102 in a flattened condition. It is noted that the embodiments shown in FIGS. 3 and 4 depict a rather close relation of the top and bottom surfaces, but this relationship can be selected as appropriate at the time of manufacture, and taking account of the desired atmosphere (if any) within the interior of the flexible sleeve 100. Although not shown, a cross-sectional view of the embodiment of FIG. 2 would be substantially similar to that obtained from FIG. 1.

In accordance with various embodiments of the present invention, the flexible sleeve 102, 202 is made of a flexible material. In one embodiment of the invention, the flexible sleeve 102, 202 can be provided by a non-woven fabric material such as a plastic-containing a non-woven fabric which is pliable yet resistant to stretching. It is also envisioned that use of a transparent plastic or other suitable polymer material which has sufficient properties including flexibility and non-elasticity may be used. It is considered that there are advantages to using a transparent or semi-transparent material to assist the surgeon in proper orientation of the implant 104 within the interior of the flexible sleeve 102, 202. Suitable transparent materials may include Mylar® brand of plastics, plastics made from Tygon® brand of plastics, vinyls, polyvinyl chloride, and other similar materials.

In one embodiment, suitable materials which are both flexible and transparent include compositions of ethylene and alpha-olefin copolymers such as the compositions used in IV saline bags. Suitable multilayer films and sealed structures are taught in U.S. Pat. Nos. 6,743,523 and 7,267,885 both of which are incorporated herein by reference for their teachings relating to suitable films for IV bags, which may also be useful with the present invention, and which may be consulted for additional details. As disclosed therein, suitable films heat sealed around their entire periphery to form suitable containers, are transparent with minimal hazing, and can be sterilized using gas sterilization or heat with intact seals and remain sufficiently flexible and pliable for the necessary manipulation described herein.

As noted, in accordance with embodiments of the present invention, the delivery systems 100, 200 can be provided in a sterile condition, with the implant 104 already inside the flexible sleeve 102. Accordingly, the material from which the flexible sleeve is made should be capable of withstanding at least one of several conventional sterilization techniques such as a steam autoclave, chemical gas sterilization, or irradiation.

In one embodiment, the interior surface of the flexible sleeve 102, 202 is provided with a low coefficient of friction to facilitate passage of the implant 104 through the flexible sleeve 102, 202 at the time the implant is to be transferred into the patient's surgical cavity.

In one embodiment, a surgically appropriate lubricant can be either included within the flexible sleeve 102, 202 at the time of manufacture, or can be injected into the interior of the flexible sleeve 102, 202 just before or during the surgical procedure in which the implant 104 will be squeezed out of the flexible sleeve for placement into the surgical pocket. The lubricant will facilitate passage of the implant 104 through the interior of the flexible sleeve. Such lubricants, if placed into the flexible sleeve during manufacture, should be sufficiently stable or inert to the sterilization conditions applied to the flexible sleeve 102, 202 and the implant 104 as part of the manufacture process. It is noted that other materials that may be present or injected into the flexible sleeve can act as a lubricant, and thus it may not be necessary to add a separate lubricant.

Since the size of the implant 104 may vary in a range from about 100 cc to about 800 cc, or larger for other types of implants, the plurality of lines 110 can be used to estimate an appropriate location that the flexible sleeve 102, 202 is to be cut to form an opening to accommodate and allow passage of various size implants 104. It is noted that the indicia 110 are schematically depicted in FIGS. 1 and 2, and there may be additional such indicia on the exterior of the flexible sleeve 102, 202 to allow the cutting of the sleeve to the proper dimensions for the size of the implant 104.

In one embodiment, the flexible sleeve 102, 104 is provided with an inner liner (not shown) which may be present within the interior of the flexible sleeve 102, 202. Such a liner may be integral with the interior surface of the flexible sleeve 102,202 or may be a separate layer of material inserted within the interior of the sleeve 102, 202 at the time of manufacture.

As described above, in various embodiments, the flexible sleeve 102, 202 further includes a valve configured for insertion of a fluid into the flexible sleeve.

As described above, in one embodiment the flexible sleeve 102, 202 includes one or more indicia formed across the first end, the second end or both the first end and the second end, the indicia configured to indicate sites at which the flexible sleeve is to be cut to form an opening from which the implant is squeezed out of the flexible sleeve into a surgical pocket during a surgical procedure. In various embodiments, locations of the indicia correlate to size of the implant.

As described above, in various embodiments, the flexible sleeve 102, 202 is transparent or semi-transparent.

As described above, in various embodiments, the delivery system 100, 200 and its contents are sterilized during manufacture.

In various embodiments, the implant is a breast implant. In one embodiment, the breast implant is a prefilled silicone breast implant. In another embodiment, the breast implant is a saline breast implant in a flattened condition and configured to be filled with saline. Such breast implants are known in the art, and are not further described herein for that reason and for brevity.

As described above, in some embodiments, the flexible sleeve further includes in its interior with the implant one or more of an antibiotic solution, an antibacterial, a lubricant, a gas, water, and/or a saline or other solution.

As will be recognized, various embodiments of the present invention are applicable to silicone breast implants. When embodiments of the present invention are used for tissue expanders or saline implants, the expander or implant will need to have an appropriate fill tube attached to the expander or implant itself prior to that implant being sealed and sterilized at the manufacturing plant. As will be recognized by the skilled person, squeezing a deflated saline implant or expander out of the bag might be more difficult and post placement adjustment of the implant or expander in the pocket might be required. Alternatively, the implant or tissue expander could be partially filled either at the time of manufacture or at the time of use. Even with this additional step at the time of surgery, embodiments of the delivery system of the present invention will still reduce or eliminate exposure of the implant or expander during handling and will reduce exposure to the operating room environment as compared to current devices and procedures.

In one embodiment, the present invention further provides a method of making a delivery system for inserting an implant into a surgical pocket, including the steps of providing an implant; providing a flexible sleeve having a first end and a second end and an outer peripheral edge, wherein the flexible sleeve is tapered toward at least one of the first end and the second end, and at least a portion of the flexible sleeve is unsealed; inserting an implant into the flexible sleeve, sealing the outer peripheral edge completely around the flexible sleeve; and sterilizing the delivery system after the inserting and the sealing. Suitable materials and methods for making and sealing multilayer films suitable for use as the flexible sleeve are taught in U.S. Pat. Nos. 6,743,523 and 7,267,885, which may also be useful with the present invention, and which may be consulted for additional details on the methods.

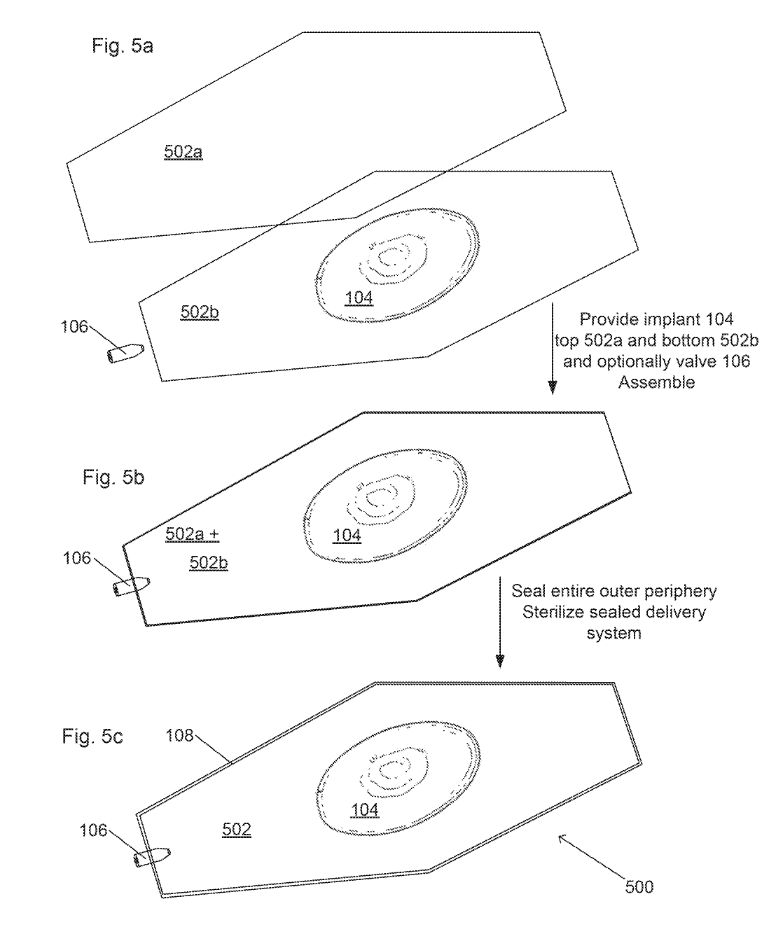

FIGS. 5a, 5b and 5c are schematic depictions of steps in a method of making a delivery system 500 for inserting an implant 104 into a surgical pocket in accordance with an embodiment of the present invention. As shown in FIG. 5a, a top panel 502a and a bottom panel 502b, each made of a suitable material in accordance with the foregoing descriptions, together with an implant 104, and, optionally, a valve 106, are provided. These items are assembled, as shown in FIG. 5b, so that the top panel 502a and the bottom panel 502b are aligned with each other, the implant 104 is located between the panels 502a and 502b, and the optional valve 106 is located, in this embodiment, in a short side of the panels. As shown in FIG. 5c, the entire outer periphery of the panels 502a and 502b are sealed, for example, by heat sealing, to form a sealed flexible sleeve 502, which is then subjected to an appropriate sterilization process to ensure that the interior of the flexible sleeve 502, including the implant 104 and the valve 106, when present, are all in a sterile condition when the delivery system 500 is provided to the surgical team in the operating room.

In one embodiment, the present invention further provides a method of inserting an implant into a surgical pocket in a patient, including the steps of providing a delivery system, the delivery system including a flexible sleeve containing an implant and having a first end and a second end, the flexible sleeve being sealed around its entire outer periphery, wherein the flexible sleeve is tapered toward at least one of the first end and the second end, and wherein the flexible sleeve is in a sterile condition at least in its interior; during a surgical procedure, cutting the flexible sleeve across at least one of the first end and the second end to form at least one opening; applying manual pressure to squeeze the implant out through one of the at least one opening and into the surgical pocket. In one embodiment, the method of inserting an implant further includes inserting one or more of an antibiotic solution, an antibacterial, a lubricant, a gas, water, or a saline or other solution into the flexible sleeve prior to the applying manual pressure step. In one embodiment, the method of inserting an implant further includes activating a lubricant that is already present in the flexible sleeve together with the implant.

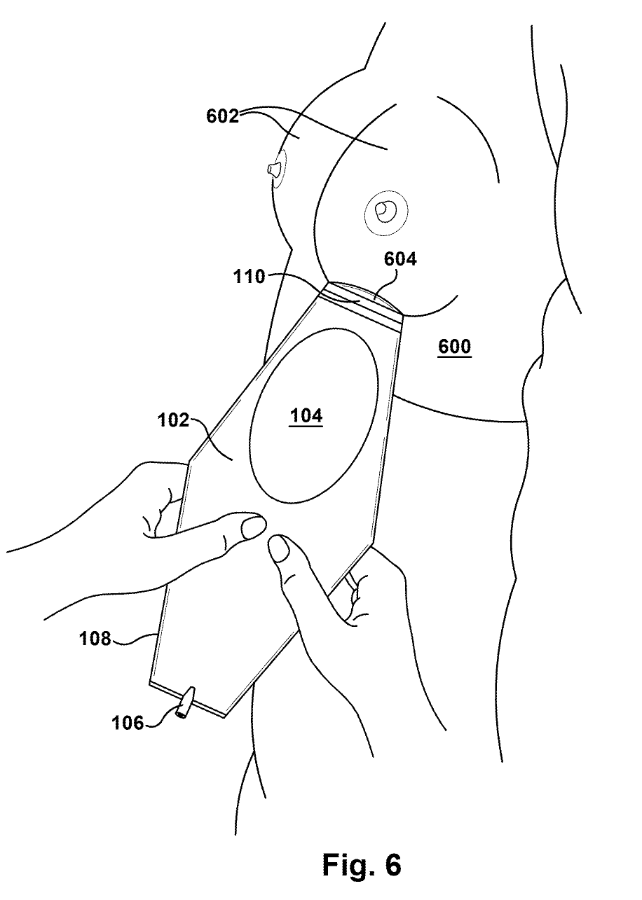

FIG. 6 is a schematic depiction of a method of inserting an implant 104 into a surgical pocket in a patient from a delivery system such as that shown in FIG. 1, in accordance with an embodiment of the present invention. As shown in FIG. 6, a patient 600, having breasts 602 is schematically depicted. The patient's left breast 602 has had an incision 604 made in the lower edge of the breast 602, to form a surgical pocket (not shown). A delivery system including a flexible sleeve 102, in accordance with an embodiment of the present invention is shown with a cut end inserted through the incision 604, so that the implant 104 can be squeezed out of the flexible sleeve 102. As shown in FIG. 6, the delivery system is substantially similar to that shown in FIG. 1, and includes a valve 106 and a sealed outer periphery 108, and indicia 110 to show optional locations for cutting the end of the flexible sleeve to an appropriate size according to the size of the implant 104, as described herein. It is noted that the embodiment of FIG. 6 illustrates the surgical incision and surgical pocket formed in the inframammary fold for use with a breast implant, but the surgical incision and surgical pocket may be formed at any other location, depending on the purpose of the implant. For breast implants, for example, the surgical incision and surgical pocket may be formed in a periaerolar or a transaxillary location. In a post-mastectomy reconstruction, the surgical incision and the surgical pocket may be larger, but the present delivery system will provide the same benefits. In one embodiment, the post-mastectomy implant is a tissue expander used to assist in stretching the patient's skin to create a suitably-sized pocket for eventual insertion of a breast implant.

The following numbered clauses provide further support for the presently disclosed and claimed invention.

Clause 1. A delivery system for inserting an implant into a surgical pocket, comprising:

a flexible sleeve having a first end and a second end and being sealed around its entire outer periphery forming an interior space, wherein the flexible sleeve is tapered toward at least one of the first end and the second end; and

an implant contained and sealed within the interior space;

wherein the interior space and the implant contained and sealed therein are in a sterile condition.

Clause 2. The delivery system of clause 1, further comprising a valve configured for insertion of a fluid into the flexible sleeve.

Clause 3. The delivery system of either of clause 1 or 2, wherein the flexible sleeve includes one or more indicia formed across the first end, the second end or both the first end and the second end, the indicia configured to indicate sites at which the flexible sleeve is to be cut to form an opening from which the implant is squeezed out of the flexible sleeve into a surgical pocket during a surgical procedure.

Clause 4. The delivery system of clause 3, wherein locations of the indicia correlate to size of the implant.

Clause 5. The delivery system of any one of clauses 1-4, wherein the flexible sleeve is transparent or semi-transparent.

Clause 6. The delivery system of any one of clauses 1-5, wherein the delivery system and its contents have been sterilized during manufacture.

Clause 7. The delivery system of any one of clauses 1-6, wherein the implant is a breast implant.

Clause 8. The delivery system of clause 7 wherein the breast implant is a prefilled silicone breast implant.

Clause 9. The delivery system of clause 7 wherein the breast implant is a saline breast implant in a flattened condition and configured to be filled with saline.

Clause 10. The delivery system of any one of clauses 1-9, wherein the flexible sleeve further comprises in its interior with the implant one or more of a lubricant, an antibiotic, an anti-bacterial composition, a gas, and water.

Clause 11. A method of making a delivery system for inserting an implant into a surgical pocket, comprising:

providing an implant;

providing a flexible sleeve having a first end and a second end and an outer peripheral edge, wherein the flexible sleeve is tapered toward at least one of the first end and the second end, and at least a portion of the flexible sleeve is unsealed;

inserting an implant into the flexible sleeve,

sealing the outer peripheral edge completely around the flexible sleeve; and

sterilizing the delivery system after the inserting and the sealing.

Clause 12. A method of inserting an implant into a surgical pocket in a patient, comprising

providing a delivery system, the delivery system comprising:

a flexible sleeve containing an implant and having a first end and a second end, the flexible sleeve being sealed around its entire outer periphery, wherein the flexible sleeve is tapered toward at least one of the first end and the second end, and wherein the flexible sleeve is in a sterile condition at least in its interior;

during a surgical procedure, cutting the flexible sleeve across at least one of the first end and the second end to form at least one opening;

applying manual pressure to squeeze the implant out through one of the at least one opening and into the surgical pocket.

It should be appreciated that the process steps and structures described below do not form a complete process flow for manufacturing items such as the flexible sleeves and delivery systems described herein. The present invention can be practiced in conjunction with fabrication techniques currently used in the art, and only so much of the commonly practiced process steps are included as are necessary for an understanding of the present invention.

While the principles of the invention have been explained in relation to certain particular embodiments, and are provided for purposes of illustration, it is to be understood that various modifications thereof will become apparent to those skilled in the art upon reading the specification. Therefore, it is to be understood that the invention disclosed herein is intended to cover such modifications as fall within the scope of the appended claims. The scope of the invention is limited only by the scope of the appended claims.

1. A delivery system for inserting an implant into a surgical pocket, comprising:
a flexible sleeve having a first end and a second end and being sealed around its entire outer periphery forming an interior space, wherein the flexible sleeve is tapered toward at least one of the first end and the second end; and
an implant contained and sealed within the interior space;
wherein the interior space and the implant contained and sealed therein are in a sterile condition.

2. The delivery system of claim 1, further comprising a valve configured for insertion of a fluid into the flexible sleeve.

3. The delivery system of claim 1, wherein the flexible sleeve includes one or more indicia formed across the first end, the second end or both the first end and the second end, the indicia configured to indicate sites at which the flexible sleeve is to be cut to form an opening from which the implant is squeezed out of the flexible sleeve into a surgical pocket during a surgical procedure.

4. The delivery system of claim 3, wherein locations of the indicia correlate to size of the implant.

5. The delivery system of claim 2, wherein the flexible sleeve includes one or more indicia formed across the first end, the second end or both the first end and the second end, the indicia configured to indicate sites at which the flexible sleeve is to be cut to form an opening from which the implant is squeezed out of the flexible sleeve into a surgical pocket during a surgical procedure.

6. The delivery system of claim 5, wherein locations of the indicia correlate to size of the implant.

7. The delivery system of claim 1, wherein the flexible sleeve is transparent or semi-transparent.

8. The delivery system of claim 1, wherein the delivery system and its contents have been sterilized during manufacture.

9. The delivery system of claim 1, wherein the implant is a breast implant.

10. The delivery system of claim 9 wherein the breast implant is a prefilled silicone breast implant.

11. The delivery system of claim 9 wherein the breast implant is a saline breast implant in a flattened condition and configured to be filled with saline.

12. The delivery system of claim 1, wherein the flexible sleeve further comprises in its interior with the implant one or more of a lubricant, an antibiotic, an anti-bacterial composition, a gas, and water.

13. The delivery system of claim 2, wherein the flexible sleeve further comprises in its interior with the implant one or more of a lubricant, an antibiotic, an anti-bacterial composition, a gas, and water.

14. The delivery system of claim 3, wherein the flexible sleeve further comprises in its interior with the implant one or more of a lubricant, an antibiotic, an anti-bacterial composition, a gas, and water.

15. The delivery system of claim 5, wherein the flexible sleeve further comprises in its interior with the implant one or more of a lubricant, an antibiotic, an anti-bacterial composition, a gas, and water.

16. A method of making a delivery system for inserting an implant into a surgical pocket, comprising:
providing an implant;
providing a flexible sleeve having a first end and a second end and an outer peripheral edge, wherein the flexible sleeve is tapered toward at least one of the first end and the second end, and at least a portion of the flexible sleeve is unsealed;
inserting an implant into the flexible sleeve,
sealing the outer peripheral edge completely around the flexible sleeve; and
sterilizing the delivery system after the inserting and the sealing.

17. A method of inserting an implant into a surgical pocket in a patient, comprising
providing a delivery system, the delivery system comprising:
a flexible sleeve containing an implant and having a first end and a second end, the flexible sleeve being sealed around its entire outer periphery, wherein the flexible sleeve is tapered toward at least one of the first end and the second end, and wherein the flexible sleeve is in a sterile condition at least in its interior;
during a surgical procedure, cutting the flexible sleeve across at least one of the first end and the second end to form at least one opening;
applying manual pressure to squeeze the implant out through one of the at least one opening and into the surgical pocket.
